(19) United States
(12) Patent Application Publication
(10) Pub. No.: US 2007/0250006 A1
(43) Pub. Date: Oct. 25, 2007

(54) MEASURING MOVEMENT OF AN ELONGATED INSTRUMENT

(75) Inventors: Ronald Court, Pelham, NH (US); Harold M. Aznoian, Seabrook, NH (US)

Correspondence Address: PROSKAUER ROSE LLP, ONE INTERNATIONAL PLACE, BOSTON, MA, 02110, US

(73) Assignee: Conmed Endoscopic Technologies, Inc., Billerica, MA (US)

(21) Appl. No.: 11/689,321

(22) Filed: Mar. 21, 2007

Related U.S. Application Data
(60) Provisional application No. 60/784,705, filed on Mar. 22, 2006.

(51) Int. Cl. A61B 5/00

(52) U.S. Cl. 604/117

(57) ABSTRACT

Methods and apparatus are provided for measuring movement of an elongated instrument. The apparatus including a guide adapted to receive the elongated instrument and a sensor module that includes an optical image sensor or an optical rotary encoder to sense the received elongated instrument moving within the guide.

### RELATED APPLICATION(S)

This application claims the benefit of U.S. Provisional Application No. 60/784,705, filed on Mar. 22, 2006. The entire teachings of the above application are incorporated herein by reference.

### FIELD OF INVENTION

The present invention relates generally to methods and apparatus for measuring the axial or rotational movement of an elongate instrument for use during a medical procedure.

### BACKGROUND

Catheters, esophageal probes, endoscopes, laparoscopes and other medical instruments are frequently introduced into body lumens for a variety of purposes, including imaging and interventional therapy. For many such procedures, it is necessary to accurately monitor the position of the instrument, particularly the distal end of the instrument which is remote from the entry point. For example, it is frequently necessary to know the precise location of the distal tip of an instrument in order to perform a subsequent interventional procedure, to facilitate interchange of instruments, and to track the precise location of an instrument during the course of a single procedure.

The penetration depth of elongate medical instruments has usually been monitored visually by the physician observing scale markings which have been placed on the side of the instrument. That is, the physician simply looks at the instrument at the point of entry and reads the approximate penetration depth from the scale. While this approach has the advantage of simplicity, it can suffer from limitations that restrict its effectiveness in modern medical procedures.

For example, the accuracy of penetration which can be determined is limited by the lack of a fixed location against which to read the scale. The accuracy is further limited by the relatively broad spacing between scale markings which are required to permit visual reading. The visual reading of the scale further requires that the physician turn away from other areas where attention should be directed. Each reading which is obtained requires additional time to be recorded and becomes obsolete as soon as the device is moved in any fashion.

### SUMMARY

According to one aspect of the invention, an apparatus is featured for measuring movement of an elongated instrument.

According to a first embodiment, the apparatus includes a guide adapted to receive the elongated instrument; a rotatable element positioned to cooperate with the guide and configured to rotate in response to axial movement of the elongated instrument within the guide; and a sensor module that includes an optical image sensor arranged in relation to the guide. The optical image sensor (a) captures images of the rotatable element as the rotatable element rotates in response to movement of the elongated instrument within the guide, (b) tracks microscopic surface features of the rotatable element across a set of the captured images, and (c) generates an indication of movement of the instrument based on the tracked microscopic surface features.

The apparatus can further include a counter that determines displacement of the instrument based on the indication of movement generated by the sensor module and a scaler that converts the displacement to an indication of movement in standard units. The apparatus can further include a housing comprising a disposable component and a fixed component. The disposable component includes the guide and the rotatable element, and the fixed component including the sensor module. The guide of the apparatus can further include an adjustable guide ceiling adapted to urge the elongated instrument against the rotatable element as the instrument moves within the guide. The adjustable guide ceiling can be adapted to rise or fall to accommodate instruments of varying dimensions.

According to a second embodiment, the apparatus for measuring movement of an elongated instrument, includes a guide adapted to receive the elongated instrument and a sensor module that includes an optical image sensor arranged in relation to the guide. The optical image sensor (a) captures images of the elongated instrument within the guide, (b) tracks microscopic surface features of the instrument across a set of the captured images, and (c) generates an indication of movement of the instrument based on the tracked microscopic surface features.

The apparatus can further include a counter that determines displacement of the instrument based on the indication of movement generated by the sensor module and a scaler that converts the displacement to an indication of movement in standard units. The apparatus can further include a housing comprising a disposable component and a fixed component. The disposable component includes the guide, and the fixed component includes the sensor module. The apparatus can further include an adjustable guide ceiling adapted to urge against the elongated instrument as the instrument moves within the guide. The adjustable guide ceiling can be adapted to rise or fall to accommodate instruments of varying dimensions.

According to a third embodiment, the apparatus for measuring movement of an elongated instrument includes a guide adapted to receive an elongated instrument; a rotatable element positioned to cooperate with the guide and configured to rotate in response to a received elongated instrument moving within the guide; and a sensor module that includes an optical rotary encoder arranged to be rotatably connected to the rotatable element. The optical rotary encoder generates an indication of movement of the instrument based on sensed rotation of the rotatable element in response to the movement of the elongated instrument within the guide.

The apparatus can further include a counter that determines displacement of the instrument based on the indication of movement generated by the sensor module and a scaler that converts the displacement to an indication of movement in standard units. The apparatus can further include a housing comprising a disposable component and a fixed component. The disposable component includes the guide and the rotatable element, and the fixed component includes the sensor module. The guide of the apparatus can further include an adjustable guide ceiling adapted to urge the elongated instrument against the rotatable element as the instrument moves within the guide. The adjustable guide ceiling can be adapted to rise or fall to accommodate instruments of varying dimensions.

According to another embodiment, the apparatus for measuring movement of an elongated instrument, includes a housing with a disposable component and a fixed component. The disposable component includes a guide adapted to receive an elongated instrument, and the fixed component includes a sensor module adapted to sense the received elongated instrument moving within the guide and to generate an indication of movement of the instrument.

According to another aspect of the invention, a method is featured for measuring movement of an elongated instrument within a guide.

According to a first embodiment, the method for measuring movement of an elongated instrument within a guide involves a rotable element being positioned to cooperate with the guide such that it rotates in response to the axial movement of the elongated instrument within the guide. The method includes the steps of capturing images of the rotatable element as the rotatable element rotates in response to movement of the elongated instrument within the guide; tracking microscopic surface features of the rotatable element across a set of the captured images; and generating an indication of movement of the instrument based on the tracked microscopic surface features.

According to a second embodiment, the method for measuring movement of an elongated instrument within a guide includes the steps of capturing images of the elongated instrument within the guide; tracking microscopic surface features of the elongated instrument across a set of the captured images; and generating an indication of movement of the instrument based on the tracked microscopic surface features.

According to a third embodiment, the method for measuring movement of an elongated instrument within a guide involves a rotatable element being positioned to cooperate with the guide, such that the rotatable element rotates in response to the axial movement of the elongated instrument within the guide. The method includes the steps of generating an indication of movement based on sensed rotation of the rotatable element in response to movement of the elongated instrument within the guide.

According to another aspect, the invention features an apparatus for measuring movement of an elongated instrument. The apparatus includes a first component including a sensor module and a second component being removably attached to the first component. The second component includes a guide adapted to receive the elongated instrument. The sensor module of the first component is arranged in relation to the guide in the second component so that the sensor module is capable of detecting movement of the elongated instrument within the guide. The second component of apparatus can be disposable, such that it can be replaced with a third component that is capable of being removably attached to the first component and includes another guide adapted to receive the elongated instrument.

### BRIEF DESCRIPTION OF DRAWINGS

FIG. 1A is a diagram illustrating an example of a measuring device for use during medical procedures.

FIG. 1B is a diagram of an endoscope to which embodiments of the measuring device can be applied.

FIG. 2 is a diagram illustrating functional components of a measuring device.

FIG. 3 is an exploded view of a first embodiment of the measuring device.

FIGS. 4A through 4E are diagrams illustrating a channel base according to the first embodiment of the measuring device.

FIGS. 5A and 5B are diagrams illustrating the sensor module according to the first embodiment of the measuring device.

FIG. 6 is a diagram illustrating the channel base according to a second embodiment of the measuring device.

FIG. 7 is a diagram illustrating a third embodiment of the measuring device that includes an optical rotary encoder.

FIG. 8 is a timing diagram illustrating the output of a particular optical rotary encoder according to the third embodiment of the measuring device.

FIG. 9 is a diagram illustrating an optional adjustable guide ceiling for use in any embodiment of the measuring device.

FIG. 10 is a diagram illustrating an optional disposable component for use in any embodiment of the measuring device.

### DETAILED DESCRIPTION

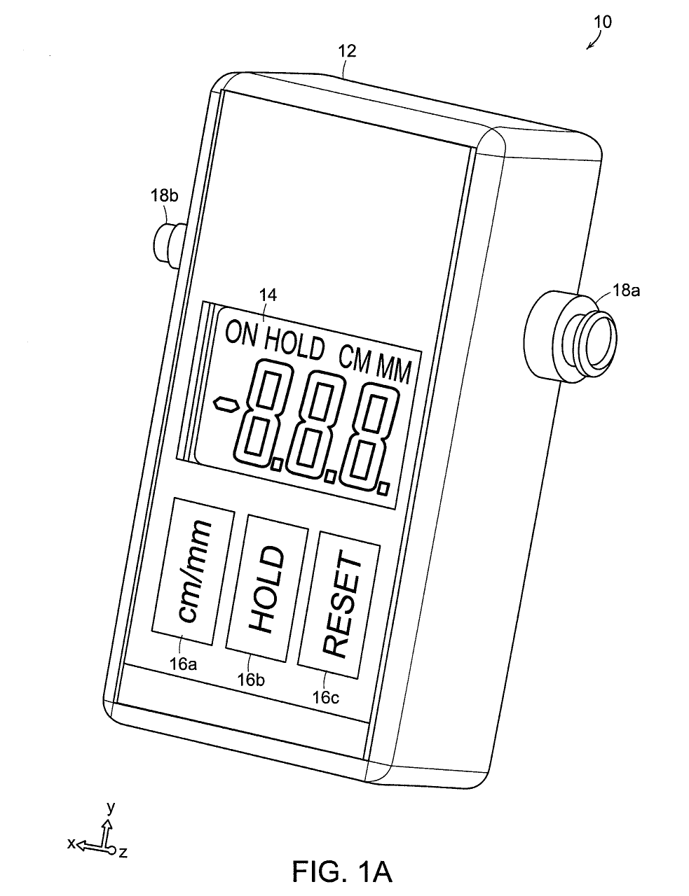

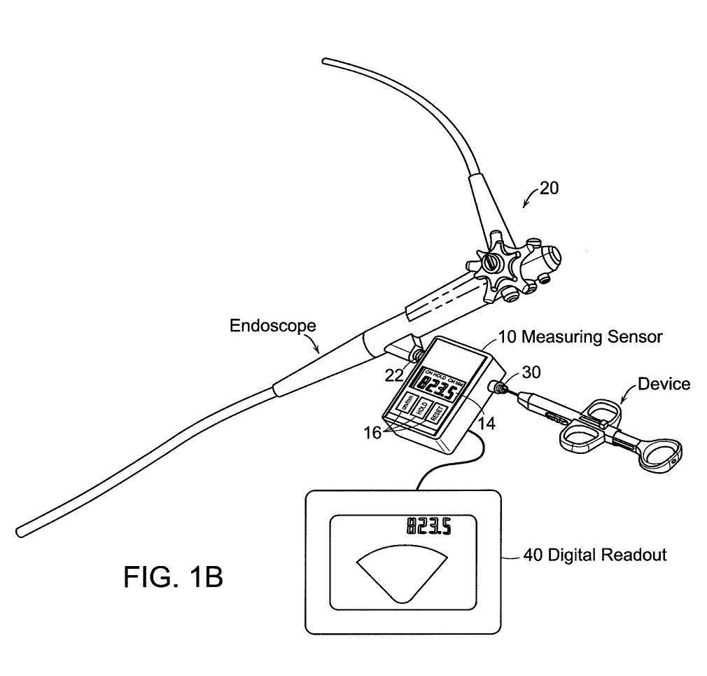

FIG. 1A is a diagram illustrating an example of a measuring device for use during medical procedures. The measuring device 10 includes a housing 12, display 14, function switches 16a, 16b, and 16c (collectively 16). A channel inlet 18a and channel outlet 18b are positioned on opposing sides of the housing 12. In operation, an elongated instrument (not shown) enters the housing through the channel inlet 18a and exits the housing 12 through the channel outlet 18b. The device 10 can be used to measure axial movements of an instrument as it is advanced or withdrawn; rotational movement of an instrument as the instrument is rotated clockwise or counter clockwise; or combinations of both axial and rotational movement of an elongated instrument.

The measuring device can include function switches 16 corresponding to the functions of unit conversion 16a, hold 16b, and reset 16c. With the unit conversion function switch 16a, an operator can change the units of the displayed measurements. For example, the measuring device 10 can convert between centimeters (cm) and millimeters (mm). Similarly, the device 10 can convert between degrees and radians. Other unit conversions are also possible. The hold function switch 16b enables an operator of the device to temporarily stabilize the displayed measurement, enabling the operator to properly record the measurement, for example. The reset function switch 16c enables the operator to zero the displayed measurement and thus reset the set point from which the measurements are made.

FIG. 1B is a diagram of an endoscope to which embodiments of the measuring device can be applied. Although the measuring device can be utilized with an endoscope, the measuring device can be utilized in other applications in which measurements of axial or rotational movement of an elongated instrument within any type of duct are desired. Likewise, the structural configuration of the measuring device can be modified to accommodate various applications. For example, the measuring device can be integrated or otherwise embedded into an endoscope itself or other similar scope. The measuring device can be fixed to a stationary platform such as a table. Other arrangements of the measuring device in relation to the lumen or other duct being measured can also be employed.

As illustrated in FIG. 1B, the endoscope 20 includes an opening 22 into a working channel through which an elongated instrument 30 can pass. The measuring device 10 can be detachably coupled to the endoscope by inserting and locking the channel outlet 18b of the device to the opening 22 of the working channel. For example, the channel outlet 18b can include a separate locking component (not shown) to lock the channel outlet into the working channel of an endoscope. Other locking mechanisms known to those skilled in the art for detachably coupling a device or instrument to the opening of a working channel can be implemented for detachably coupling the measuring device to the endoscope. The elongated instrument 30 enters into the channel inlet 1 8a, through the device 10, and out the channel outlet 18b into the working channel of the endoscope. As the instrument 30 is advanced, withdrawn, or rotated a corresponding axial or rotational measurement can be presented on the display 14.

In practice, the measuring device 10 is capable of determining penetration depth of an instrument or the dimensions of any tissue sample. For example, the measuring device 10 can be used to measure the distance between the distal end of the endoscope 20 to a targeted tissue sample (not shown). This can be performed by advancing the endoscopic instrument 30 through the device 10 into the working channel of the endoscope. Once the instrument 30 reaches the distal end of the endoscope 20, the operator can zero the displayed measurement by depressing the Reset switch 16c. From this reconfigured set point, the operator can continue advancing the instrument 30 until it reaches the targeted sample. The displayed measurement is the distance from the distal end of the endoscope 20 to the targeted sample.

An operator of the measuring device 10 can also determine the dimensions of a targeted tissue sample, such as a polyp or stone for example. This sizing function can be performed by advancing the endoscopic instrument 30 until the targeted sample is reached, resetting the displayed measurement of the measuring device 10 by depressing the Reset switch 16c, and advancing or withdrawing the instrument along the body of the tissue sample to obtain its length for display.

In addition to displaying the real-time measurements through display 14 of the measuring device 10, signals indicative of the real-time measurements can be coupled to a video monitor 40 for display. Such real-time measurement can overlay a corresponding video display of the distal end of the instrument 30 being operated within a field of interest.

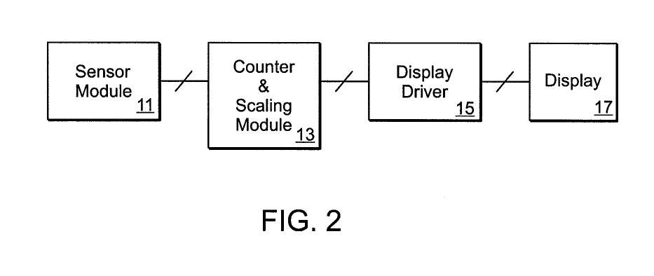

FIG. 2 is a diagram illustrating functional components of a measuring device. The functional components include a sensor module 11, a counter and scaling module 13, a display driver 15, and a display 17. The sensor module 11 detects movement of an instrument and provides one or more signals indicative of the amount and direction of the detected movement to the counter and scaling module 13. The counter and scaling module 13 receives the one or more signals and generates an accumulated value, or count, representing the net displacement of the instrument. The counter and scaling module 13 scales this count to a measurement value in desired units based on predetermined ratios of counts to desired units. This measurement value is then transmitted to the display driver 15, which causes the display 17 to present the measurement value, preferably in real time. The measurement values can also be output to an external peripheral, such as a monitor (not shown).

The sensor module can be implemented in a number of different ways to measure, or otherwise determine, the axial or rotational movement of an instrument. According to a first embodiment, the sensor module includes an optical image sensor that detects movement of the instrument indirectly by analyzing a sequence of captured images of a rotatable element that is in contact with the elongated instrument. According to a second embodiment, the sensor module includes an optical image sensor that detects movement of an instrument directly by analyzing a sequence of captured images of the instrument itself as it passes in view of the image sensor. According to a third embodiment, the sensor module includes an optical rotary encoder that detects movement of the instrument indirectly by coupling the encoder to a rotatable element in contact with the instrument. A number of optional features can be applied to each of these embodiments for further enhancement as described more fully below.

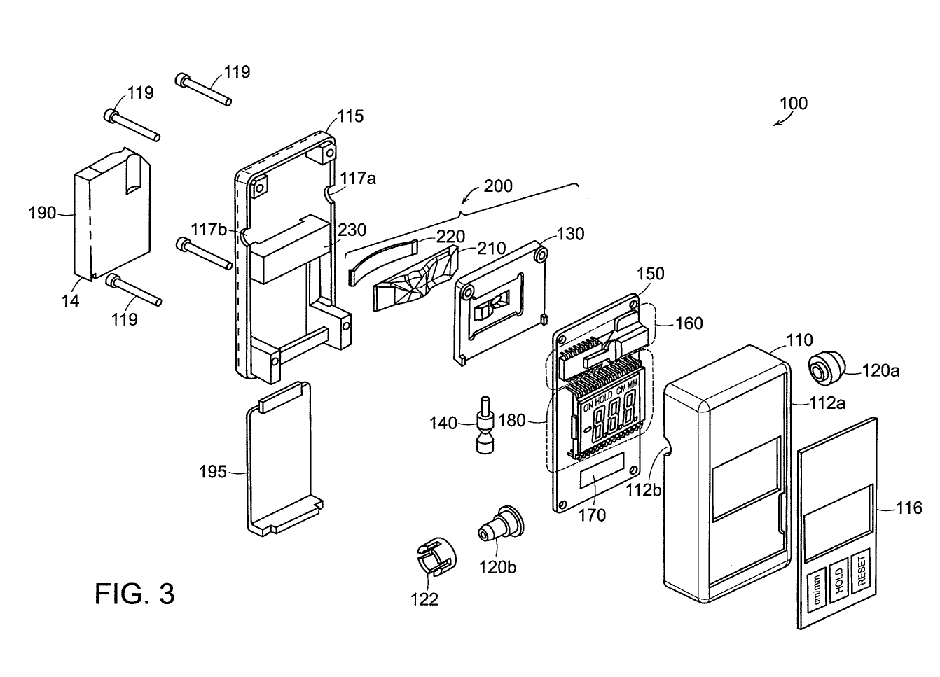

FIG. 3 is an exploded view of a first embodiment of the measuring device. In the illustrated embodiment, the housing of the measuring device 100 includes a sensor front cover 110 and a sensor back cover 115. The front cover 110 further includes a set of membrane switches 116 that can be depressed to trigger predefined functionality, such as unit conversion, hold and reset. Both the front and back covers each define notches in the sidewalls 112a, 112b and 117a, 117b, respectively, into which the channel inlet 120a and channel outlet 120b are fixed. The channel outlet 120b can further include a channel lock 122 that locks the channel outlet 120b into the working channel of an endoscope. For example, as the channel outlet 120b is inserted into the working channel, the channel lock 122 extends over and engages the opening of the working channel. Other locking mechanisms known to those skilled in the art for detachably coupling a device or instrument to the opening of a working channel can be implemented for detachably coupling the measuring device to the endoscope.

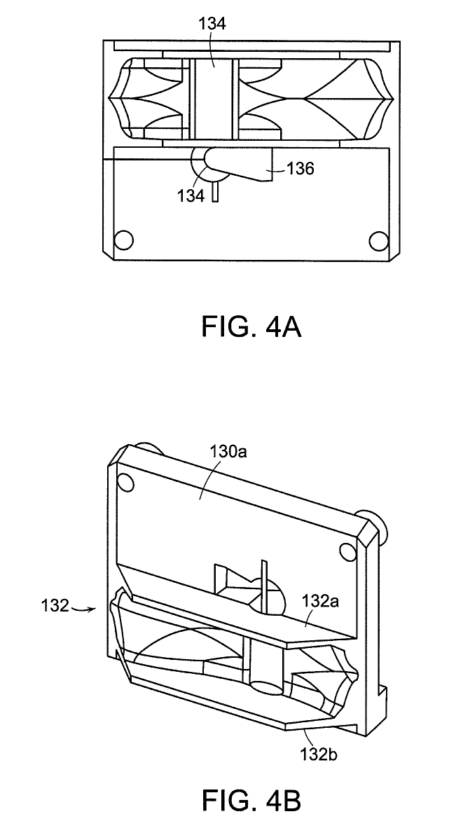

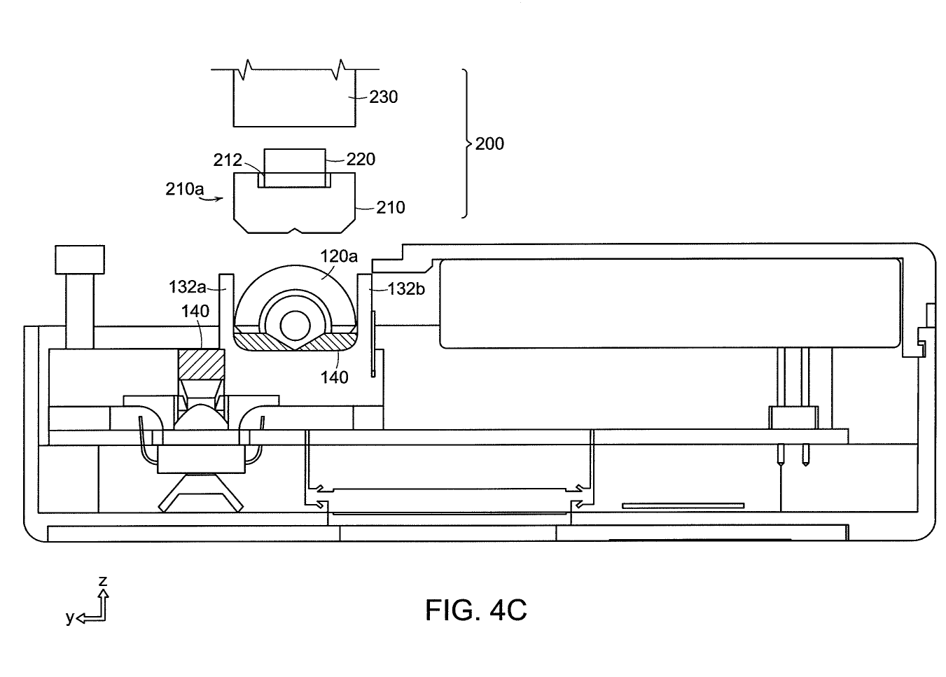

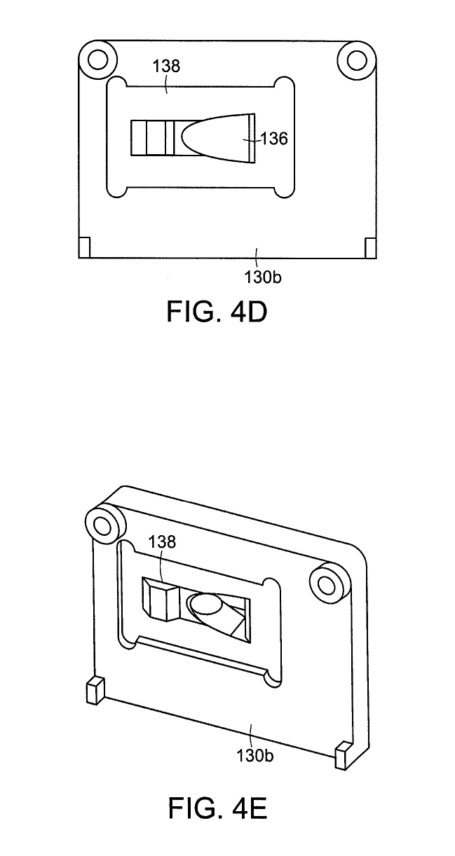

As the elongated instrument is inserted into the housing via the channel inlet 120a, a channel base 130 guides the instrument toward the channel outlet 120b. FIGS. 4A through 4E are diagrams illustrating a channel base according to the first embodiment of the measuring device. In FIGS. 4A and 4B, a guide 132 is formed on an outer surface of the channel base 130a including a pair of sidewalls 132a, 132b extending from the base. The surface of the base 130 within the guide 132 can also be contoured to provide for self-centering of the instrument as it passes through the guide. A depression 134 is formed in the channel base 130 extending at least between the sidewalls of the guide 132.

Referring to FIG. 4C, a roller 140 is positioned within the depression 134 of the channel base. As shown, the roller 140 projects through at least one of the sidewalls 132a, 132b external to the guide. As an instrument enters the guide, the instrument is urged against the roller 140 by an adjustable guide ceiling 200. Optionally, the instrument can be urged against the roller using a fixed ceiling, another rotatable element or other opposing element. As the instrument continues to be advanced or withdrawn, the roller 140 rotates in a clockwise or counter-clockwise direction depending on the movement of the instrument. The roller 140 can be replaced with a ball bearing, cylinder, or other rotatable element known to those skilled in the art, for example.

Referring to FIGS. 4D and 4E, a through hole 136 is defined in the channel base 130 such that the roller 140 is exposed to a processing module positioned adjacent the opposing side of the base 130b.

Referring back to FIG. 3, the processing module 150 includes a sensor module 160, a counter and scaling module 170 and an LCD display 180. The sensor module 160 is aligned adjacent to the surface 130b of the channel base. The sensor module 160 captures and processes images of the exposed surface of the roller 140 to determine the corresponding axial movement of the elongated instrument within the guide.

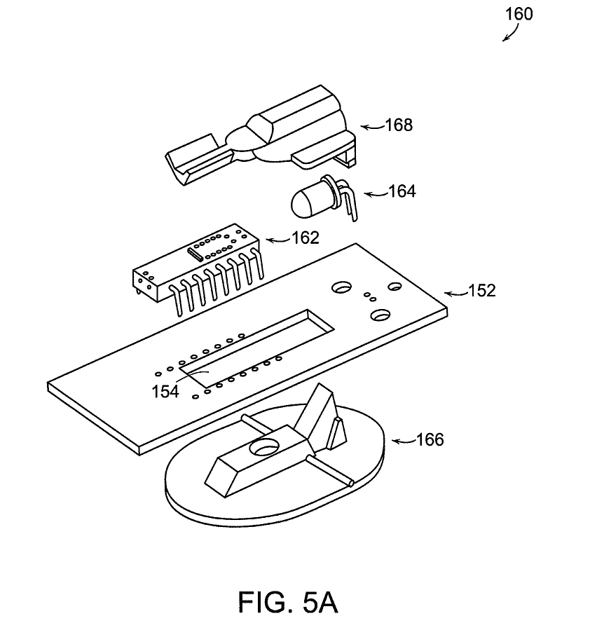

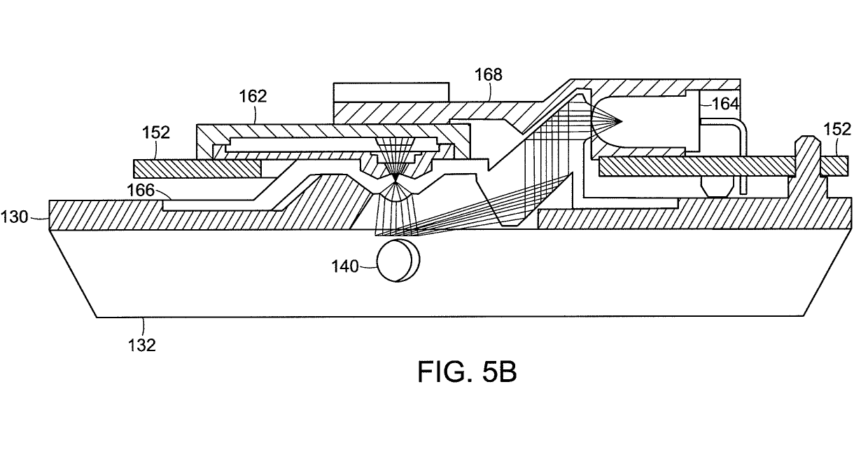

FIGS. 5A and 5B are diagrams illustrating the sensor module according to the first embodiment of the measuring device. The sensor module 160 includes an optical image sensor 162, a light source 164, a lens 166, and a clip 168. The sensor 162 is mounted on a printed circuit board (PCB) 152 above a through hole 154 defined in the board. The light source 164, such as a Light Emitting Diode (LED), is also mounted on the board 152 and interlocked to the sensor 162 with the clip 168. The lens 166 is aligned below the sensor 162 through the hole defined in the board 154. The clip 168 also holds the LED 164 in relation to the lens 166.

As shown in FIG. 5B, the sensor module 160 is aligned to the channel base 130 directly above the exposed surface of the roller. An alignment projection 138 extending from a surface of the base plate, as shown in FIG. 4B, can assist in alignment of the channel base 130 through hole 154 to the sensor module 160. Light from the LED 164 is reflected through the lens 166 via the openings in the board 152 and the channel base 130 to illuminate the exposed surface of the roller 140 below.

The sensor 162 focused through the lens 166 detects movement of the roller 140 by capturing images of the roller 140 as it turns. From these captured images, the optical sensor 162 detects microscopic features on the surface of the roller 140 in the images and tracks their movement across a set of frames. The amount and direction of the tracked movement corresponds to movement of the instrument passing through the channel guide 132. The sensor 162 encodes the amount and direction of the tracked movement and transmits the encoded data to the counter and scaling module.

For improved detection of surface feature detection, the roller 140 is manufactured such that its outer surface or portion thereof is optically irregular. For example, the roller can be manufactured out of a material that is capable of providing an inherently optically irregular surface (e.g., ceramics). The roller can also be manufactured such that the material is processed so that its outer surface is textured or otherwise roughened to provide an optically irregular surface. The roller can also be manufactured such that the roller or portion thereof is covered with another textured or roughened materials to provide such an irregular surface (e.g., rubber made coarse through the application of sandpaper). Other ways of manufacturing a roller or other rotatable element with an irregular optical surface can be applied that are known to those skilled in the relevant arts.

Examples of suitable components for this embodiment include the Agilent ADNS-2030 Low Power Optical Mouse Sensor for the optical sensor 162; the HDNS-2100 for the lens 166; the HDNS-220 for the clip 168, and the HLMP-ED80-xx000 LED for the light source 164; all from Avago Technologies having co-headquarters in San Jose, Calif. and Singapore. For more information regarding these components of the sensor module, refer to data sheet entitled “Agilent ADNS-2030 Low Power Optical Mouse Sensor,” the entire contents of which are incorporated herein by reference.

Referring back to FIG. 3, the sensor 162 interfaces to the counter and scaling module 170. The sensor 162 transmits the amount and direction of the tracked movement to the counter and scaling module 170 through one or more pulsed signals. For example, the Agilent ADNS-2030 Low Power Optical Mouse Sensor encodes the amount and direction of movement in a form of quadrature output. The quadrature output includes two pulsed signals that, in combination, represent both the amount and direction of tracked movement. As the instrument is moved in a first direction, the pulsed signals cycle through a predetermined sequence of states (e.g., 00, 01, 11, 10). Each change in state corresponds to a count, and a number of counts can be defined per measurement (e.g., centimeters, millimeters, radians, degrees, inch, etc). Conversely, as the instrument is moved in the opposing direction, the sequence of states continue in reverse, thus enabling the counter and scaling module 170 to detect a change in direction and increment/decrement the accumulated number of pulse counts accordingly. The counter and scaling module 170 accumulates the pulse counts and converts them into a measurement using a predetermined ratio of counts to desired units. The resulting measurement is then transmitted for presentation through the LCD display 180. The counter and scaling module 170 can be implemented, for example, using a suitably programmed or dedicated processor (e.g., a microprocessor or microcontroller), hardwired logic, Application Specific Integrated Circuit (ASIC), and a Programmable Logic Device (PLD) (e.g, Field Programmable Gate Array (FPGA)).

According to a second embodiment of the measuring device, movement of the instrument is sensed directly by analyzing a sequence of captured images of the instrument itself as it passes in view of an optical image sensor. This second embodiment of the measuring device can be implemented using the same sensor module as the first embodiment by modifying the channel base of the first embodiment such that the surface of the instrument itself passes within the view of the sensor module.

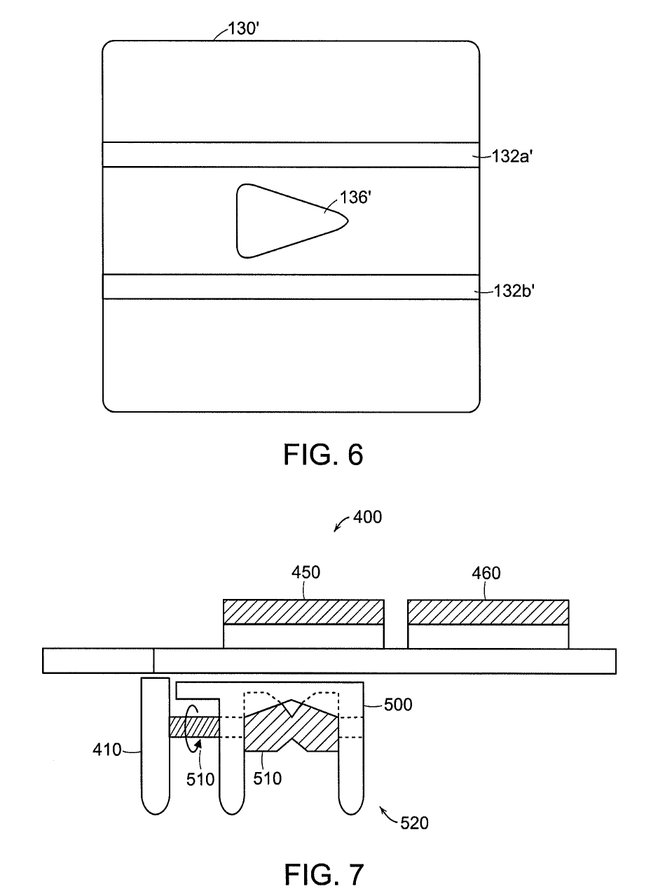

FIG. 6 is a diagram illustrating the channel base according to the second embodiment of the measuring device. In particular, the channel base 130′ is modified to omit the roller and defines a through hole 136′ in a space between the sidewalls 132a′, 132b′ of the guide, such that the surface of the instrument is exposed to the opposing side of the base. Thus, as the elongated instrument traverses through or rotates within the guide 132′, light emitted from the LED 164 is reflected through the lens 166 through openings 154, 136′ in the board and channel base, respectively, to illuminate the surface of the instrument.

The sensor 162, which is aligned to the channel base 130′, captures images focused through the lens 166 of the exposed portion of the instrument as it moves within the channel guide 132′. From these captured images, the optical sensor 162 detects microscopic features on the surface of instrument in the images and tracks their movement across a set of frames along one or more axes (e.g., X-axis, Y-axis). The amount and direction of the tracked movement along X-axis corresponds to axial movement of the instrument being advanced or withdrawn. The amount and direction of the tracked movement along the Y-axis corresponds to rotation movement of the instrument within the guide. The sensor 162 encodes the amount and direction of the tracked movement along each axis and transmits the encoded data to the counter and scaling module 170 as described above in connection with the first embodiment. Subsequent processing and display is also similar to the first embodiment.

According to a third embodiment of the measuring device, movement of the instrument is sensed indirectly by coupling an optical rotary encoder to a rotatable element that is in contact with the instrument. This third embodiment of the measuring device can be implemented using a different type of sensor module that includes an optical rotary encoder by modifying the processing module 150 and the channel base 130 of the first embodiment.

For example, FIG. 7 is a diagram illustrating a third embodiment of the measuring device that includes an optical rotary encoder. The processor module 400 includes a sensor module comprised of an optical rotary encoder 410, a counter and scaling module 450 and a display 460. The channel base 500 is similar to the base of the first embodiment, including a roller 510 or other rotatable element being rotatably connected to the optical rotary encoder 410. As an instrument enters the guide 520, the instrument is urged against the roller 510 by an adjustable or fixed ceiling or other opposing element causing the roller to rotate in a clockwise or counter-clockwise direction, depending on the direction of the axial movement of the instrument. In turn, the roller 510 rotatably engages the rotary encoder 410, which converts the rotary motion of the roller into a linear measurement of predefined units, referred to herein as “counts”.

According to particular embodiments, the optical rotary encoder 410 is implemented using Quick Assembly Two and Three Channel Optical Encoders HEDM-5500/5600, HEDS-5500/5540, and HEDS-5600/5640; all from Avago Technologies, Inc. with co-headquarters in Palo Alto, Calif. and Singapore. The outputs of the HEDS-5500/5600 and HEDM-5500/5600 are two square waves in quadrature (CH.A and CH.B). The HEDS-5540 and 5640 can also have a third channel index output (CH.I) which is generated once for each full rotation of the codewheel in addition to the two channel quadrature (CH.A and CH.B). Standard resolutions between 96 and 1024 counts per revolution are presently available for these encoders. For more information regarding these components, refer to their technical data sheet entitled “Quick Assembly Two and Three Channel Optical Encoders,” the entire contents of which are incorporated herein by reference.

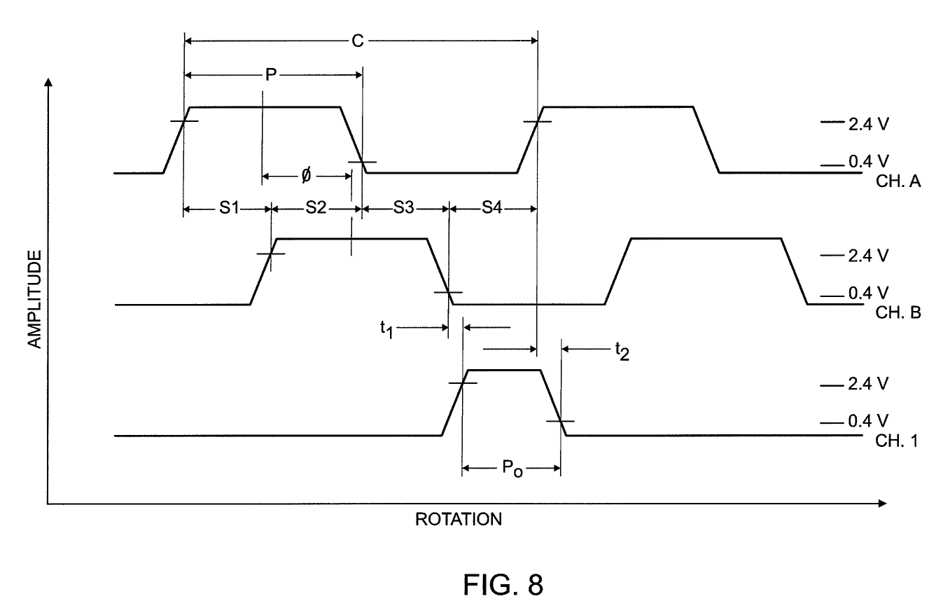

FIG. 8 is a timing diagram illustrating the output of a particular optical rotary encoder according to the third embodiment of the measuring device. Specifically, exemplary waveforms are shown for the output signals on channels CH.A, CH.B and CH.I of the identified Avago optical encoders. Each pulse corresponds to a count. The resolution of the encoder depends on the number of counts per revolution. When the roller 510 engages the rotary encoder in the counterclockwise direction channel CH.A leads channel CH.B. Conversely, if the roller 510 engages the rotary encoder in the clockwise direction channel CH.B leads channel CH.A. Thus, the phase difference between channels CH.A and CH.B can be used to detect whether the direction of instrument movement within the guide. The total count in a particular direction can be translated or scaled to the axial or rotational movement in a corresponding direction.

The signal outputs of channels CH.A and CH.B are transmitted to the counter and scaling module 450, which maintains an accumulated total number of counts and increments/decrements the accumulated count depending on the direction of the rotary motion. Assuming a particular number of counts per unit measurement (e.g, centimeters, millimeters, inches, etc.), the counter and scaling module 450 can convert the accumulate value into desired units of measurement for presentation through the display 460. The resulting measurement represents movement of the instrument passing through the guide.

Advantages of above-described embodiments include the ability to detect the amount and direction of instrument movement regardless of whether the elongated instrument includes optical marks and high accuracy and resolution.

Optionally, any of the above-described embodiments can be further enhanced with an adjustable guide ceiling that enables instruments of different dimensions to pass through the guide of the channel base. For example, as shown in FIG. 3 of the first embodiment of the measuring device, an adjustable guide ceiling 200 is provided including a channel finger 210, a channel sprint 220, and a back support 230. This adjustable ceiling is capable of rising and falling to accommodate different sized instruments.

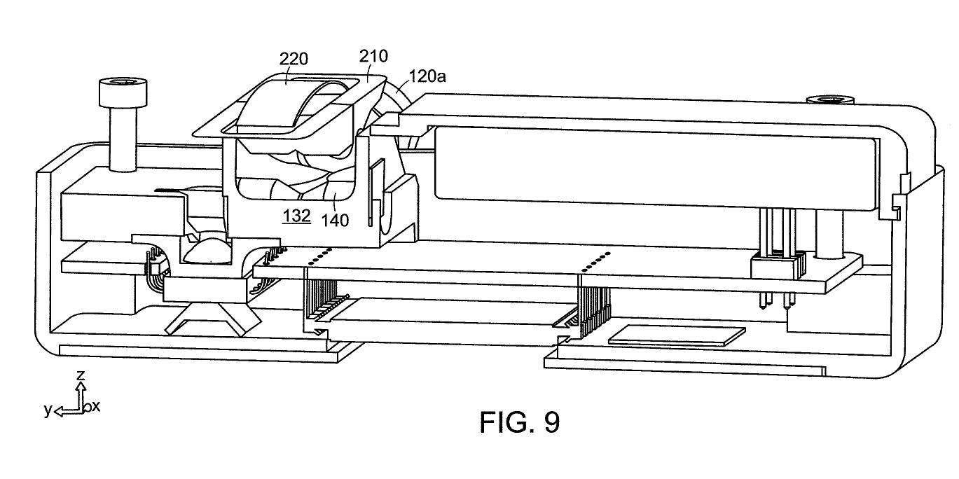

FIG. 9 is a diagram illustrating an optional adjustable guide ceiling for use in any embodiment of the measuring device. In this illustrated embodiment, the channel finger 210 is loosely positioned within the sidewalls of the guide 132. The channel spring 220 is formed as an arch having ends fixedly attached to the back support 230 that is located within the sensor back cover 115. The channel spring 220 exerts a force on the channel finger such that it is urged into the guide 132. According to one embodiment, the channel finger is constructed such that when the spring is in its maximally extended position, there is a minimal clearance between a lower surface of the channel finger 21 Oa and the roller 140.

In operation, as an elongated instrument is inserted into the channel guide 132, an opposing force of the instrument causes the channel finger 210 to rise and push back on the channel spring 220. As a result, the inserted instrument is urged against roller 140 causing it to turn. Preferably, the spring constant of the channel spring should be selected to minimize the resistance felt by the operator of the device as the instrument is inserted. Springs having different spring constants can be implemented to accommodate alternate ranges of instrument sizes.

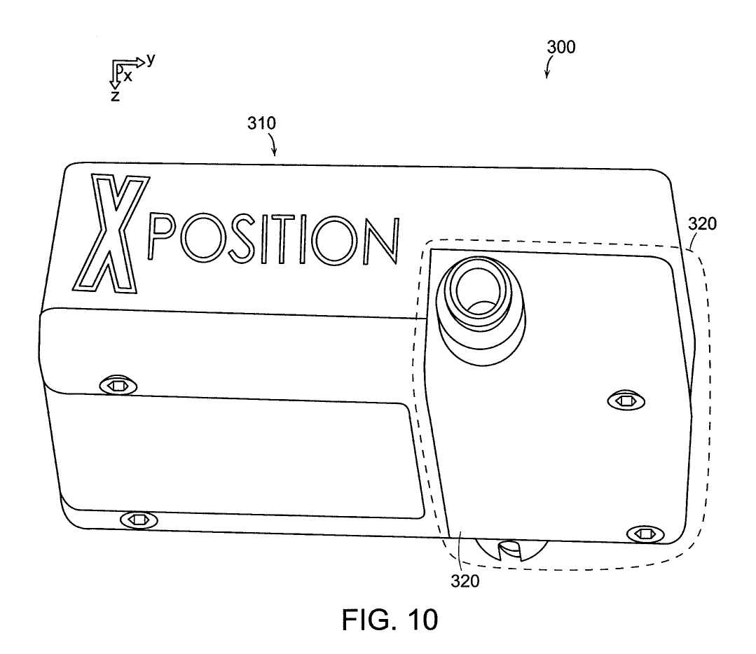

Optionally, a disposable component may be incorporated into any of the above-described embodiments. For example, FIG. 10 is a diagram illustrating an optional disposable component for use in any embodiment of the measuring device. In this embodiment, the measuring device 300 includes a fixed component 310 and a disposable component 320. The fixed component 310 includes at least a sensor module. The disposable component, which is removably attached to the fixed component, includes at least a guide adapted to receive the elongated instrument. The sensor module of the fixed component is arranged in relation to the guide in the disposable component so that the sensor module is capable of detecting movement of the elongated instrument within the guide, for example, as described above in connection with any of the foregoing embodiments.

The disposable component can be removably attached to the fixed component using any suitable means known to one skilled in the relevant arts. For example, the disposable component can “snap” in and out of the fixed component; the disposable component can slide into and out a receptor of the fixed component; the disposable component can be attached to the fixed component using a fixing mechanism such as a screw or bolt, for example; the fixed component can include a locking mechanism to receive the disposable component in connection with the fixed component and a release mechanism to unlock or otherwise release the disposable component from the fixed component.

The disposable component 320 contains at least those constituent components which are likely to become contaminated due to the advancement and withdrawal of an elongated instrument into a body lumen. For example, with respect to the first embodiment of the measuring device, such constituent components can include the channel base 130, channel finger 210, channel spring 220, back support 230, roller 140, lens 166, channel inlet 120a, channel outlet 120b and channel lock 122 as described in FIG. 3. The fixed housing component 310 contains the remaining portions of the device including the battery 190, the battery cover 195, the processing module 150 and its constituent components excluding the lens 166, as shown and described with respect to FIG. 3. The actual positioning and dimensions of the constituent components within the fixed and disposable components can be modified such that the disposable component 320 can be readily detached from the fixed component 310. In this way, the disposable component can be replaced with another disposable component containing the same or a different set of device components depending on the application or instruments to be measured.

While this invention has been particularly shown and described with references to preferred embodiments thereof, it will be understood by those skilled in the art that various changes in form and details may be made therein without departing from the scope of the invention encompassed by the appended claims.

What is claimed:

1. An apparatus for measuring movement of an elongated instrument, comprising:
a guide adapted to receive the elongated instrument;
a rotatable element positioned to cooperate with the guide, the rotatable element configured to rotate in response to axial movement of the elongated instrument within the guide; and
a sensor module that includes an optical image sensor arranged in relation to the guide, wherein the optical image sensor (a) captures images of the rotatable element as the rotatable element rotates in response to movement of the elongated instrument within the guide, (b) tracks microscopic surface features of the rotatable element across a set of the captured images, and (c) generates an indication of movement of the instrument based on the tracked microscopic surface features.

2. The apparatus of claim 1 further comprising:
a counter that determines displacement of the instrument based on the indication of movement generated by the sensor module;
a scaler that converts the displacement to an indication of movement in standard units.

3. The apparatus of claim 1 further comprising:
a housing comprising a disposable component and a fixed component, the disposable component including the guide and the rotatable element, the fixed component including the sensor module.

4. The apparatus of claim 1 wherein the guide further comprises:
an adjustable guide ceiling adapted to urge the elongated instrument against the rotatable element as the instrument moves within the guide, the adjustable guide ceiling adapted to rise or fall to accommodate instruments of varying dimensions.

5. An apparatus for measuring movement of an elongated instrument, comprising:
a guide adapted to receive the elongated instrument; and
a sensor module that includes an optical image sensor arranged in relation to the guide, wherein the optical image sensor (a) captures images of the elongated instrument within the guide, (b) tracks microscopic surface features of the instrument across a set of the captured images, and (c) generates an indication of movement of the instrument based on the tracked microscopic surface features.

6. The apparatus of claim 5 further comprising:
a counter that determines displacement of the instrument based on the indication of movement generated by the sensor module;
a scaler that converts the displacement to an indication of movement in standard units.

7. The apparatus of claim 5 further comprising:
a housing comprising a disposable component and a fixed component, the disposable component including the guide, the fixed component including the sensor module.

8. The apparatus of claim 5 wherein the guide further comprises:
an adjustable guide ceiling adapted to urge against the elongated instrument as the instrument moves within the guide, the adjustable guide ceiling adapted to rise or fall to accommodate instruments of varying dimensions.

9. An apparatus for measuring movement of an elongated instrument, comprising:
a guide adapted to receive an elongated instrument;
a rotatable element positioned to cooperate with the guide, the rotatable element configured to rotate in response to a received elongated instrument moving within the guide; and
a sensor module that includes an optical rotary encoder arranged to be rotatably connected to the rotatable element, wherein the optical rotary encoder generates an indication of movement of the instrument based on sensed rotation of the rotatable element in response to the movement of the elongated instrument within the guide

10. The apparatus of claim 9 further comprising:
a counter that determines displacement of the instrument based on the indication of movement generated by the sensor module;
a scaler that converts the displacement to an indication of movement in standard units.

11. The apparatus of claim 9 further comprising:
a housing comprising a disposable component and a fixed component, the disposable component including the guide and the rotatable element, the fixed component including the sensor module.

12. The apparatus of claim 9 wherein the guide further comprises:
an adjustable guide ceiling adapted to urge the elongated instrument against the rotatable element as the instrument moves within the guide, the adjustable guide ceiling adapted to rise or fall to accommodate instruments of varying dimensions.

13. An apparatus for measuring movement of an elongated instrument, comprising:
a housing including a disposable component and a fixed component, the disposable component including a guide adapted to receive an elongated instrument, the fixed component including a sensor module adapted to sense the received elongated instrument moving within the guide and to generate an indication of movement of the instrument.

14. A method for measuring movement of an elongated instrument within a guide, a rotable element being positioned to cooperate with the guide, such that the rotatable element rotates in response to the axial movement of the elongated instrument within the guide, the method comprising:
capturing images of the rotatable element as the rotatable element rotates in response to movement of the elongated instrument within the guide;
tracking microscopic surface features of the rotatable element across a set of the captured images; and
generating an indication of movement of the instrument based on the tracked microscopic surface features.

15. A method for measuring movement of an elongated instrument within a guide, comprising:
capturing images of the elongated instrument within the guide;
tracking microscopic surface features of the elongated instrument across a set of the captured images; and
generating an indication of movement of the instrument based on the tracked microscopic surface features.

16. A method for measuring movement of an elongated instrument within a guide, a rotatable element being positioned to cooperate with the guide, such that the rotatable element rotates in response to the axial movement of the elongated instrument within the guide, comprising:
generating an indication of movement based on sensed rotation of the rotatable element in response to movement of the elongated instrument within the guide.

17. An apparatus for measuring movement of an elongated instrument, comprising:
a first component including a sensor module; and
a second component being removably attached to the first component, the second component including a guide adapted to receive the elongated instrument;
wherein the sensor module of the first component is arranged in relation to the guide in the second component so that the sensor module is capable of detecting movement of the elongated instrument within the guide.

18. The apparatus of claim 17 wherein the second component is disposable, such that the second component can be replaced with a third component that is capable of being removably attached to the first component and includes another guide adapted to receive the elongated instrument.
